SEQUENCE LISTING (6 sequences)

SEQ ID NO: 1 - DNA, 35 bp, Homo sapiens
ggcgagctcactttttctgtcacctccgtggtccg

SEQ ID NO: 2 - DNA, 28 bp, Homo sapiens
gggttcgaaacggcggcggcggtggaga

SEQ ID NO: 3 - DNA, 20 bp, Homo sapiens
ttttcatcgtcgtccctagc

SEQ ID NO: 4 - DNA, 20 bp, Homo sapiens
cgactcagatgtggcagaaa

SEQ ID NO: 5 - DNA, 19 bp, Homo sapiens
gaaggtgaaggtcggagtc

SEQ ID NO: 6 - DNA, 20 bp, Homo sapiens
gaagatggtgatgggatttc

(19) United States
(12) Patent Application Publication
(10) Pub. No.: US 2011/0212176 A1
(43) Pub. Date: Sep. 1, 2011

(54) CICLOPIROX AND CYTARABINE FOR THE TREATMENT OF LEUKEMIC DISORDERS

(75) Inventors: Aaron D. Schimmer, Thornhill (CA); Yanina Eberhard, Toronto (CA)

(73) Assignee: UNIVERSITY HEALTH NETWORK, Toronto, ON (CA)

(21) Appl. No.: 13/126,941

(22) PCT Filed: Oct. 29, 2009
(86) PCT No.: PCT/CA2009/001546

§ 371 Date: Apr. 29, 2011

Related U.S. Application Data
(60) Provisional application No. 61/110,075, filed on Oct. 31, 2008.

(51) Int. Cl. A61K 31/7068; A61P 35/02; A61K 9/28

(52) U.S. Cl. 424/474; 514/49

(57) ABSTRACT

The present application relates to compositions and methods for treating leukemic disorders. In particular, the present application relates to compositions and methods for treating the leukemic disorders such as acute myeloid leukemia (AML) and acute lymphoid leukemia (ALL) using ciclopirox and cytarabine.

### FIELD

The application relates to methods and compositions for the treatment of leukemic disorders and particularly to methods and compositions for the treatment of leukemic disorders such as acute myeloid leukemia (AML) or acute lymphoid leukemia (ALL) in a subject.

### BACKGROUND

Acute myeloid leukemia (AML) and acute lymphoid leukemia (ALL) are leukemic disorders resulting in the proliferation of abnormal cells of myeloid and lymphoid origin, respectively. Both diseases are characterized by poor responses to standard therapies. For example, elderly patients with either AML or ALL and poor risk cytogenetics have a median survival of less than one year. Thus, for these patients and those with relapsed refractory disease novel therapies are needed. As many of these patients are frail, therapies that achieve an anti-leukemia effect without significant toxicity are highly desirable.

Ciclopirox olamine (CPX) is a broad-spectrum antifungal agent used in the treatment of a variety of fungal and yeast infections of the skin. It inhibits the growth of fungi through its ability to bind intracellular iron. However, its anti-cancer effects are essentially unknown.

### SUMMARY

Described herein is a novel treatment for leukemic disorders. Ciclopirox (CPX) induces cell death in leukemia cells and a combination treatment using CPX and cytarabine has synergetic effects. It has also been shown that CPX reduces tumor size in an in vivo mouse model.

Accordingly, in one aspect the present application describes a method for treating a leukemic disorder comprising administering an effective amount of CPX and an effective amount of cytarabine to a subject in need of such treatment. In one embodiment, the leukemic disorder is acute myeloid leukemia. In a further embodiment the leukemic disorder is acute lymphoid leukemia. In yet a further embodiment, the leukemic disorder is chronic myelogenous leukemia. In a further embodiment, the chronic myelogenous leukemia comprises chronic myelogenous leukemia in blast crises. In a further embodiment, the leukemic disorder is a lymphoma. In yet a further embodiment the leukemic disorder is multiple myeloma.

A further aspect is a method of treating acute leukemia, for example acute myeloid leukemia, comprising administering an effective amount of CPX and an effective amount of cytarabine to a subject in need of such treatment.

In certain embodiments, the effective amount of CPX is within the range of about 1 to about 200 mg/kg body weight with an effective amount of cytarabine. In one embodiment, the effective amount is within the range of about 5 to about 50 mg/kg body weight of CPX and an effective amount of cytarabine.

Another aspect is use of an effective amount of CPX and an effective amount of cytarabine for the treatment of a leukemic disorder.

A further aspect is use of an effective amount of CPX and an effective amount of cytarabine in the preparation of a medicament for the treatment of a leukemic disorder.

In certain embodiments, the use of an effective amount of CPX and an effective amount of cytarabine is for the treatment of acute myeloid leukemia.

In certain embodiments, the use of an effective amount of CPX and an effective amount of cytarabine is for the treatment of acute lymphoid leukemia.

In certain embodiments, the use of an effective amount of CPX and an effective amount of cytarabine is for the treatment of chronic myelogenous leukemia.

In certain embodiments, the use of an effective amount of CPX and an effective amount of cytarabine is for the treatment of lymphoma or multiple myeloma. In a further aspect, the application describes methods and uses wherein the CPX and/or cytarabine are comprised in a composition described herein.

In yet a further aspect, the application describes methods and uses wherein the CPX and/or cytarabine are comprised in a dosage form described herein.

A further aspect is a pharmaceutical composition for the treatment of a leukemic disorder comprising an effective amount of CPX and an effective amount of cytarabine and a pharmaceutically acceptable carrier in a dosage form, wherein the dosage form is suitable for oral administration or injection.

A further aspect is a pharmaceutical composition for the treatment of acute myeloid leukemia in a subject, which composition comprises as active ingredients CPX and cytarabine, and a pharmaceutically acceptable carrier in unit dosage form, wherein the pharmaceutical composition is suitable for oral administration or injection.

A further aspect is a pharmaceutical composition for treatment of acute lymphoid leukemia in a subject, which composition comprises as active ingredients CPX and cytarabine, and a pharmaceutically acceptable carrier in unit dosage form, wherein the pharmaceutical composition is suitable for oral administration or injection.

A further aspect is a pharmaceutical composition, which is in an embodiment for the treatment of chronic myelogenous leukemia in a subject, which composition comprises as active ingredients CPX and cytarabine, and a pharmaceutically acceptable carrier in unit dosage form, wherein the pharmaceutical composition is suitable for oral administration or injection.

A further aspect is a pharmaceutical composition for treatment of lymphoma or multiple myeloma in a subject, which composition comprises as active ingredients CPX and cytarabine, and a pharmaceutically acceptable carrier in unit dosage form, wherein the pharmaceutical composition is suitable for oral administration or injection.

A further aspect is a composition, wherein the oral dosage form is selected from enteric coated tablets, caplets, gelcaps, and capsules, comprising from about 20 to less than about 1000 mg, suitably from about 50 to about 500 mg, of CPX and an effective amount of cytarabine.

A further aspect is a composition, wherein the tablets or capsules containing about 20 to less than about 1000 mg, suitably from about 50 to about 500 mg, of CPX and an effective amount of cytarabine.

A further aspect is a commercial package comprising a composition according to the present disclosure, and associated therewith instructions for the use thereof for treatment of a leukemic disorder such as acute myeloid leukaemia, acute lymphoid leukemia, or chronic myelogenous leukemia in a subject in need of such treatment.

A further aspect is a commercial package comprising a composition according to the present disclosure, and associated therewith instructions for the use thereof for treatment of a leukemic disorder such as lymphoma or multiple myeloma, in a subject in need of such treatment. Other features and advantages of the present application will become apparent from the following detailed description. It should be understood, however, that the detailed description and the specific examples while indicating preferred embodiments of the application are given by way of illustration only, since various changes and modifications within the spirit and scope of the application will become apparent to those skilled in the art from this detailed description.

### BRIEF DESCRIPTION OF THE DRAWINGS

An embodiment of application will now be described in relation to the drawings in which:

FIG. 1: A screening for inhibitors of survivin transactivation identifies the antifungal CPX. (A) HeLa cells stably overexpressing the survivin promoter driving luciferase were treated with increasing concentrations of CPX for 24 hours. Data represent their mean+SD percentage luciferase expression compared to cells treated with buffer control. (B) HeLa cells were treated with 5 μM CPX. At increasing times after incubation, cells were harvested and total proteins were isolated. Expression of survivin and GAPDH were measured by immunoblotting.

FIG. 2: CPX induces cell death in malignant cell lines. Leukemia and non-malignant cell lines were treated with increasing concentrations of CPX. After 72 hours of incubation, cell viability was measured by MTS assay. Data represent the mean percentage of viable cells±SD.

FIG. 3: CPX synergizes with AraC (cytarabine) to induce cell death in leukemia cells. (A) Effect of CPX and AraC (cytarabine) alone or in combination at the ratio 1:0.6 in OCI-AML2 cells represented as the fractional effect in which 1 is equal to 100% inhibition. (B) Combined effect of CPX and AraC (cytarabine) quantitatively evaluated by the CI method. CIs of <0.3, 0.3-0.7, 0.7-0.85, 0.85-0.90, 0.90-1.10 or >1.10 indicate strong synergism, synergism, moderate synergism, slight synergism, additive effect or antagonism, respectively.

FIG. 4: CPX delays tumor growth in mouse models of leukemia. Sublethally irradiated NOD/SCID mice were injected intraperitoneally with MDAY-D2 murine leukemia cells, or subcutaneously injected with K562 human leukemia cells or OCI-AML2 human leukemia cells. After implantation, mice were treated with CPX (25 mg/kg) or vehicle control by oral gavage daily. After 8 days (MDAY-D2 cells) or 30 days (K562 and OCI-AML2 cells) mice were sacrificed and tumors were excised, measured and weighted. Bars represent the median of the population±SD. Medians were compared by the Mann-Whitney non-parametric test and the p values shown.

FIG. 5: Cell cycle arrest, survivin decrease and p53 increase precede CPX induced cell death. A) HeLa, PPC-1 and MDAY-D2 cells were treated with 5 μM CPX or buffer control. Eighteen hours after incubation, cell cycle was analyzed by PI staining and Flow cytometry. B) NB4 and OCI-M2 cell lines were incubated with CPX (5 μM) for 48 hours. After incubation cells were harvested and nuclear proteins were isolated. Levels of p53 and the nuclear protein glucocorticoid receptor (GR) were measured by immunoblotting. C) Solid tumor (OVCAR3, HT-29, HepG2, DU145, HeLa) and leukemia (OCI-M2, OCI-AML2, NB4, HL-6) cell lines were treated with CPX (2.5 μM) or buffer control for 48 hours. After incubation, total protein was isolated. Levels of survivin and GAPDH were measured by immunoblotting.

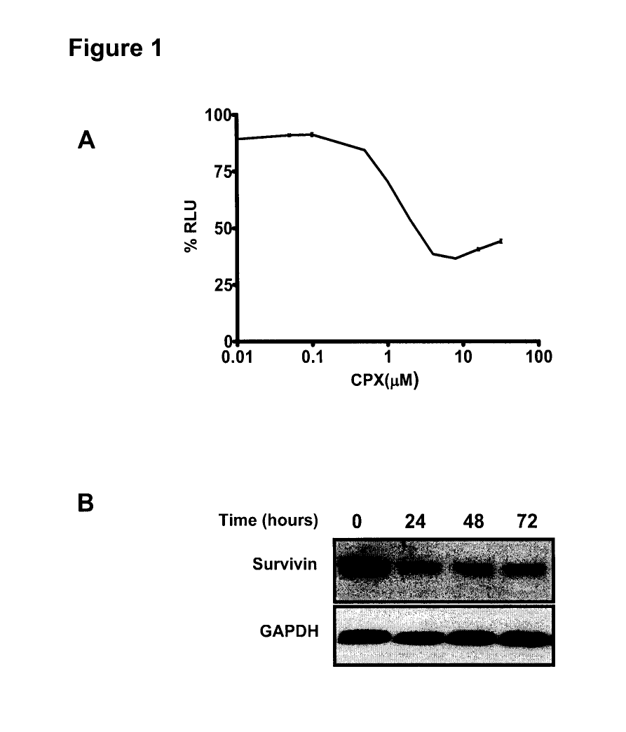

### DESCRIPTION OF VARIOUS EMBODIMENTS

### I. Definitions

The term “AraC” also referred to herein as “cytarabine” and also known as cytosine arabinoside refers to 1-(β-D-arabino-furanosyl)-cytosine and/or 4-amino-1-[(2R,3S,4R,5R)-3,4-dihydroxy-5-(hydroxymethyl)oxolan-2-yl]pyrimidin-2-one and includes all pharmaceutically acceptable salts, solvates, and prodrugs thereof as well as combinations thereof.

The term “cell death” as used herein includes all forms of cell death including necrosis and apoptosis.

The term “ciclopirox” and/or “CPX”, as used herein means 6-Cyclohexyl-1-hydroxy-4-methyl-2(1H)-pyridone and includes all pharmaceutically acceptable salts, solvates, and prodrugs thereof as well as combinations thereof. For example ciclopirox can be for example ciclopirox olamine, which comprises 6-Cyclohexyl-1-hydroxy-4-methyl-2(1H)-pyridone with 2-aminoethanol in a 1:1 ratio.

As used herein, “contemporaneous administration” and “administered contemporaneously” means that CPX and cytarabine are administered to a subject such that they are both biologically active in the subject at the same time. The exact details of the administration will depend on the pharmacokinetics of the two substances in the presence of each other, and can include administering one substance within 24 hours of administration of the other, if the pharmacokinetics are suitable. Designs of suitable dosing regimens are routine for one skilled in the art. In particular embodiments, two substances will be administered substantially simultaneously, i.e. within minutes of each other, or in a single composition that comprises both substances.

As used herein, the term “control” refers to in the context of comparing levels for example survivin levels, a suitable non-leukemic disorder cell or population of cells, including, for example cells from an individual or a group of individuals who do not have a leukemic disorder. For example, when detecting increased survivin activity or levels in a particular leukemia cell type such as AML cells, the control, can optionally be a non-cancerous myeloid cell. The control can also refer to a reference level corresponding to for example levels in a suitable non-leukemic disorder cell or population of cells. Control in the context of comparing drug efficacy, for example, in mouse experiments, refers to untreated or mock treated mice e.g. control mice and/or an administered solution not containing the compound being tested (e.g. buffer control). A person skilled in the art would readily understand the control intended by the context.

As used herein, the phrase “dosage form” refers to the physical form of a dose for example comprising compounds of the application, and includes without limitation tablets, including enteric coated tablets, caplets, gelcaps, capsules, ingestible tablets, buccal tablets, troches, elixirs, suspensions, syrups, wafers, and the like. The dosage form may be solid or liquid.

As used herein, the phrase “effective amount” or “therapeutically effective amount” means an amount effective, at dosages and for periods of time necessary to achieve the desired result. For example in the context or treating a leukemic disorder, an effective amount is an amount that for example induces remission, reduces tumor burden, and/or prevents tumor spread or growth compared to the response obtained without administration of the compound. Effective amounts may vary according to factors such as the disease state, age, sex, weight of the animal. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

As used herein, the phrase “effective amount of cytarabine” means for example an amount of cytarabine as approved by a health regulatory agency, such as the FDA, for the treatment of a subject having a leukemic disorder e.g. an amount known to be effective for treating a leukemic disorder.

The term “leukemic disorder cell” as used herein refers to a cell or cell line derived from a leukemic disorder and includes for example leukemia cells such as HL-60, RSV411, K562, Jurkat, U937, OCI-M2, OCI-AML2 and NB4 leukemia cell lines and cells phenotypically similar thereto, lymphoma cells such as MDAY-D2 and cell phenotypically similar thereto, and multiple myeloma cells such as OPM2, KMS11, LP1, UTMC2, KSM18 and OCIMy5 myeloma cell lines and cells phenotypically similar thereto. Leukemic disorder cells also include chronic myelogenous leukemia cells, including cells representing the blast crises phases such as K562 and cells phenotypically similar thereto; AML cells such as; HL-60, K562, OCI-M2, and NB4 and cells phenotypically similar thereto, ALL cells such as RSV411 and Jurkat and cells phenotypically similar thereto, and lymphoma cells such as MDAY-D2 and cells phenotypically similar thereto.

The term “leukemic disorder” as used herein means any disease involving the progressive proliferation of abnormal immature leukocytes found in hemopoietic and lymphoid tissues, other organs and usually in the blood in increased numbers. Leukemic disorder includes for example acute myeloid leukemia (AML), including promyelocytic leukemia, mixed lineage and other subtypes of AML, high-risk acute myeloid leukemia, acute lymphoid leukemia (ALL) and chronic myelogenous leukemia (CML), including for example chronic myelogenous leukemia in the chronic phase, the accelerated phase and in blast crisis. Also included are lymphomas and multiple myeloma.

As used herein, to “inhibit” or “suppress” or “reduce” a function or activity, such as survivin activity, is to reduce the level or function or activity of survivin when compared to otherwise same conditions except for a condition or parameter of interest, or alternatively, as compared to another condition. Similarly to “inhibit” or “suppress” or “reduce” expression such as survivin expression, is to reduce the level of expression when compared to otherwise same condition or parameter or interest, or alternatively as compared to another condition.

The term “pharmaceutically acceptable” means compatible with the treatment of animals, in particular, humans.

The term “pharmaceutically acceptable salt” means an acid addition salt which is suitable for or compatible with the treatment of patients.

In general, prodrugs will be functional derivatives of the compounds of the application which are readily convertible in vivo into the compound from which it is notionally derived. Prodrugs of the compounds of the application may be conventional esters formed with the available hydroxy and/or amino group. For example, the available OH and/or NH₂ in the compounds of the application may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the application are those in which the hydroxy and/or amino groups in the compounds is masked as groups which can be converted to hydroxy and/or amino groups in vivo. Conventional procedures for the selection and preparation of suitable prodrugs are described, for example, in “Design of Prodrugs” ed. H. Bundgaard, Elsevier, 1985.

The term “phenotypically similar” refers to a cell type that exhibits morphological, physiological and/or biochemical characteristics similar to another cell type. For example, a cell that is phenotypically similar to an AML cell can include a cell that comprises Auer rods. As another example, U937 cells which are derived from a patient with lymphoma, show morphological similarity to monocytoid AML cells. As a further example the leukemia cell line NB4 differentiates similar to promyelocytic cells with all trans retinoic acid (ATRA).

The term “solvate” as used herein means CPX and cytarabine or pharmaceutically acceptable salts of CPX and cytarabine, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions.

The term “subject” as used herein includes all members of the animal kingdom including mammals, and suitably refers to humans.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early stage myeloma can be treated to prevent progression or alternatively a subject in remission can be treated with a compound or composition described herein to prevent recurrence. Treatment methods comprise administering to a subject a therapeutically effective amount of a compound described in the present application and optionally consists of a single administration, or alternatively comprises a series of applications. For example, the compounds described herein may be administered at least once a week. However, in another embodiment, the compounds may be administered to the subject from about one time per week to about once daily for a given treatment. In another embodiment, the compound is administered twice daily. The length of the treatment period depends on a variety of factors, such as the severity of the disease, the age of the patient, the concentration, the activity of the compounds described herein, and/or a combination thereof. It will also be appreciated that the effective dosage of the compound used for the treatment or prophylaxis may increase or decrease over the course of a particular treatment or prophylaxis regime. Changes in dosage may result and become apparent by standard diagnostic assays known in the art. In some instances, chronic administration may be required.

It is to be understood that the terms as defined herein are intended to apply in all embodiments of the application.

### II. Method/Uses of CPX and Cytarabine

A novel therapeutic and combination treatment for treating leukemic disorders such as acute myeloid leukemia (AML) and acute lymphoid leukemia (ALL) have been identified. Using a chemical biology screen for inhibitors of survivin transactivation, the screen surprisingly identified the anti-parasitic compound ciclopirox (CPX) as being an inhibitor of survivin transactivation.

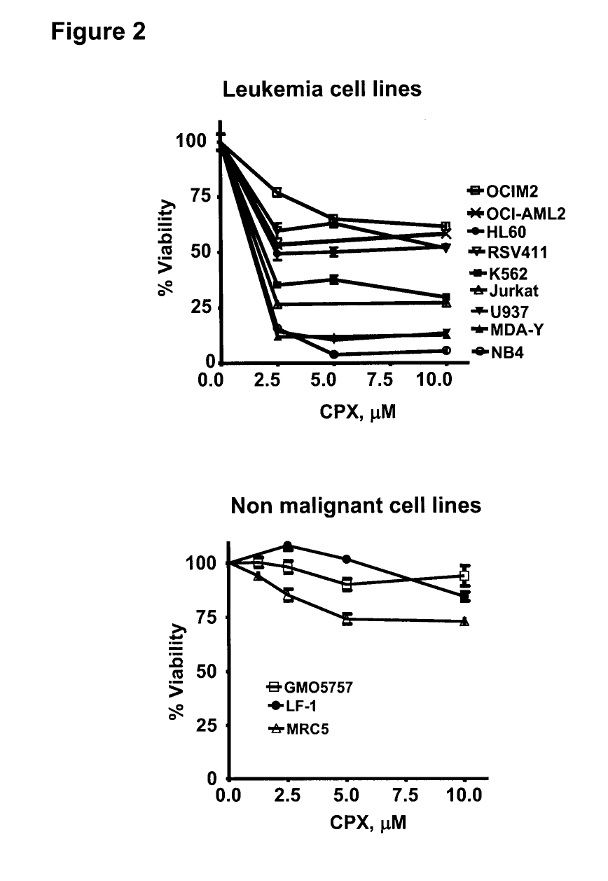

To explore its efficacy and mechanism of action as an anti-cancer agent, leukemia cell lines were treated with increasing concentrations of ciclopirox. Seventy two hours after incubation, cell viability was measured by the MTS assay. CPX decreased cell viability in all leukemia and lymphoma cell lines and decreased cell viability in 5/9 leukemia/lymphoma cell lines with an LD₅₀<5 μM (FIG. 2), a concentration that is pharmacologically achievable based on prior animal studies investigating CPX as an anti-fungal. Cell death was confirmed by the presence of a subG1 peak by flow cytometry after staining cells with propidium iodide. In contrast, CPX was less toxic to MRC 5, LF1, and GMO 5757 non-malignant fibroblasts with an LD₅₀>20 μM (FIG. 2).

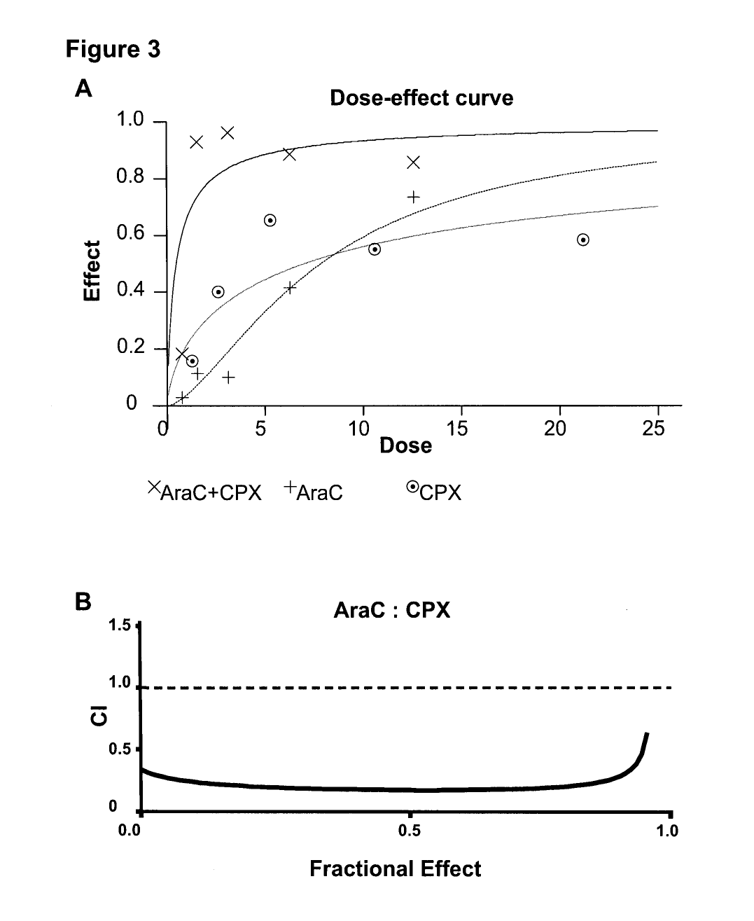

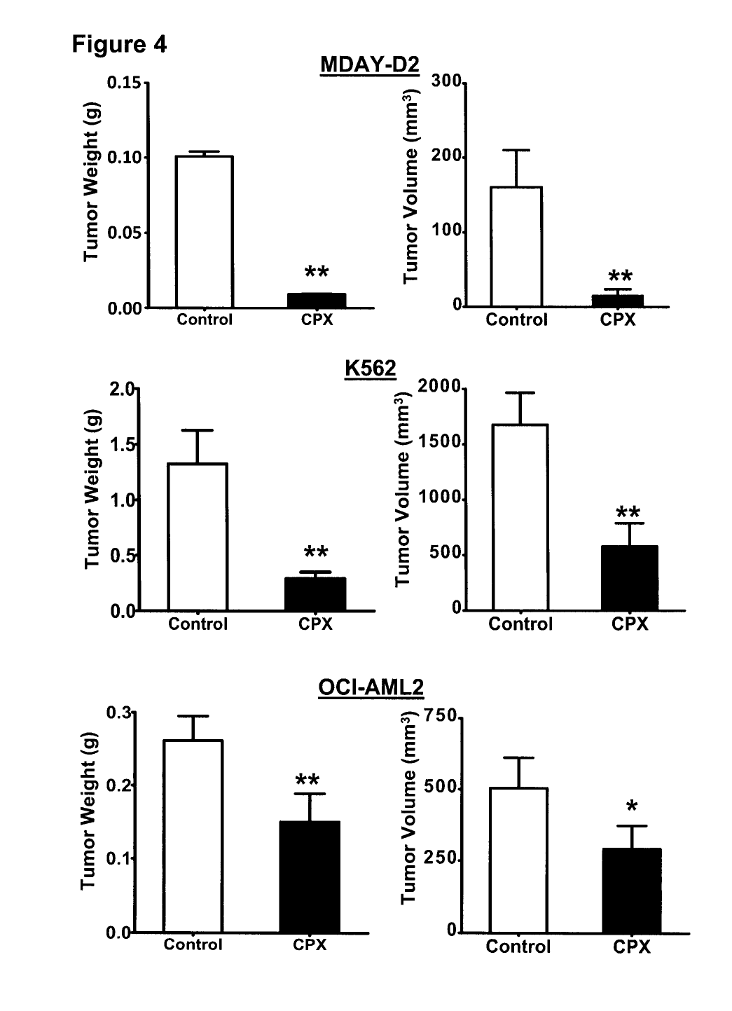

Next, CPX was evaluated in combination with cytarabine. In AML cell lines CPX synergistically enhanced the cytotoxicity of cytarabine as determined by the median effect isobologram analysis. Specifically, the combination indices (CI) at the EC50, 75 and 90 were 0.18, 0.19, and 0.24, respectively, where a CI<0.9 denotes synergy (FIG. 3). In contrast, the addition of CPX to daunorubicin produced only additive effects. Given the effects in leukemia cells lines, the effects of oral CPX was evaluated in 3 mouse models of leukemia/lymphoma. Sublethally irradiated NOD-SCID mice were injected subcutaneously with OCI-AML2 or K562 human leukemia cells or intraperitoneally with MDAY-D2 murine lymphoma cells. After tumor implantation, mice were treated with CPX (25 mg/kg) in water or water alone by oral gavage. Oral CPX decreased tumor weight and volume in all 3 mouse models by up to 65% compared to control without evidence of weight loss or gross organ toxicity (FIG. 4).

It is known that cytarabine shows clinical activity in lymphoma and myeloma as well as leukemia. Leukemias, lymphomas, and myelomas are all hematologic malignancies (e.g leukemic disorders). AML cells are generally derived from cells of the myeloid lineage, lymphomas, while other leukemias and myelomas are derived from lymphoid lineage. However, myeloid and lymphoid lineage cells derive from a common hematopoietic precursor. Based on the teachings herein, it is expected that leukemic disorders including leukemias, lymphomas and myelomas are treatable by an effective amount of CPX and an effective amount of cytarabine.

Accordingly, the present application describes a method of treating leukemia by administering an effective amount of CPX and an effective amount of cytarabine to a subject in need of such a treatment. The present application also provides use of an effective amount of CPX and an effective amount cytarabine for the treatment of leukemic disorders, including leukemias, lymphomas and myelomas.

In a further aspect, the application describes methods and uses wherein the CPX and/or cytarabine are comprised in a composition described herein.

In yet a further aspect, the application describes methods and uses wherein the CPX and/or cytarabine are comprised in a dosage or dosage form described herein.

The dosage administered will vary depending on the use and known factors such as the pharmacodynamic characteristics of the particular substance, and its mode and route of administration, age, health, and weight of the individual recipient, nature and extent of symptoms, kind of concurrent treatment, frequency of treatment, and the effect desired. Dosage regime may be adjusted to provide the optimum therapeutic response.

In certain embodiments, the leukemic disorder is an acute leukemia. In one embodiment the acute leukemia is acute myeloid leukemia. In another embodiment, the acute leukemia is acute lymphoid leukemia. In a further embodiment, the leukemia is a high risk acute leukemia. In another embodiment, the leukemia is chronic myelogenous leukemia. In a further embodiment, the leukemic disorder is lymphoma. In a further embodiment, the leukemic disorder is multiple myeloma. In yet a further embodiment, the leukemic disorder is a refractory malignancy.

In one embodiment, the subject in need thereof has leukemia. In another embodiment, the subject has acute myeloid leukemia. In another embodiment, the subject has high-risk acute myeloid leukemia. In one embodiment the leukemia is acute lymphoid leukemia. In another embodiment, the leukemia is chronic myelogenous leukemia. In a further embodiment, the subject has lymphoma. In a further embodiment, the subject has multiple myeloma. In a further embodiment, the subject has a refractory malignancy.

Without wishing to be bound by theory, the mechanism of CPX and cytarabine action may involve one or more of the following pathways. As mentioned, survivin over-expressed in multiple myeloma (MM) and a subset of high-risk patients with acute myeloid leukemia (AML) or acute lymphoid leukemia (ALL), contributing to their pathogenesis and chemoresistance (1). Further inhibition of survivin by antisense oligonucleotides has been shown to have preclinical efficacy in models of malignancy (2). As it has been shown herein that CPX reduces survivin expression, CPX may be affecting one or more of these pathways.

It has also been demonstrated that CPX and CPX in combination with cytarabine induces cell death in leukemic disorder cells. Accordingly, the application describes a method of inducing cell death in a leukemic disorder cell or population of leukemic disorder cells comprising contacting the cell or cells with CPX and cytarabine, wherein the combined amount of CPX and cytarabine is sufficient to induce cell death.

In one embodiment CPX and cytarabine induce cell death in at least 20%, at least 30%, at least 40%, at least 50%, at least 60%, at least 70%, or at least 80%, at least 90%, at least 95% or more of cells.

In another embodiment, the application describes a method of inducing cell death in a leukemic disorder cell or population of leukemic disorder cells comprising administering an effective amount of CPX and cytarabine to the cell or cells.

In one embodiment, the cell is or is phenotypically similar to an acute myeloid leukemia cell. In another embodiment, the cell is or is phenotypically similar to an acute lymphoid leukemia cell. In another embodiment, the cell is phenotypically similar to a chronic myelogenous leukemia cell. In other embodiments, the cell is phenotypically similar to a lymphoma or multiple myeloma cell.

As mentioned, it has been found that CPX reduces survivin levels. Accordingly, an aspect provides a method of inhibiting survivin activity or survivin levels in a cell comprising contacting the cell with CPX or with CPX and cytarabine, wherein the CPX or CPX and cytarabine are provided in an amount effective to decrease the activity or level of survivin in the cell. In another embodiment, the method comprises detecting increased activity or level of survivin in a leukemic disorder cell or population of leukemic disorder cells, compared to a control; and contacting the cell or cells with CPX or with CPX and cytarabine, wherein the CPX or CPX and cytarabine are provided in an amount effective to decrease the activity or level of survivin in the cell or population of cells.

In one embodiment, the decrease in the level of survivin and/or the activity of survivin is at least 15%, at least 20%, at least 30%, at least 40%, at least 50%, at least 60%, at least 70% at least 80%, or more than 80%.

In certain embodiment, the cell or population of cells is in vivo.

In one embodiment, CPX is administered or contacted with the cell prior to administering or contacting the cell with cytarabine. In another embodiment, cytarabine is administered or contacted with the cell prior to administering or contacting the cell with CPX. In a further embodiment, CPX and cytarabine are administered or contacted with the cell contemporaneously.

A person skilled in the art will recognize that the methods and uses described herein can also be combined with other therapies known in the art.

### III. Compositions

An aspect of the application provides a composition comprising CPX and cytarabine. In an embodiment the composition comprising CPX and cytarabine is for treating leukemic disorders such as leukemia lymphoma or multiple myeloma. In one embodiment, the composition is a pharmaceutical composition. In another embodiment, the composition comprises an effective amount of CPX. In a further embodiment, the composition comprises an effective amount of cytarabine.

It is to be clear that the present application describes pharmaceutically acceptable salts, solvates and prodrugs of CPX and cytarabine and mixtures comprising two or more of CPX and cytarabine, pharmaceutically acceptable salts of CPX and cytarabine, pharmaceutically acceptable solvates of CPX and cytarabine and prodrugs of CPX and cytarabine.

The compounds may have at least one asymmetric centre. Where the compounds described herein possess more than one asymmetric centre, they may exist as diastereomers. It is to be understood that all such isomers and mixtures thereof in any proportion are encompassed within the scope of the present application. It is to be understood that while the stereochemistry of the compounds of the application may be as provided for in any given compound listed herein, such compounds of the application may also contain certain amounts (e.g. less than 20%, suitably less than 10%, more suitably less than 5%) of compounds of the application having alternate stereochemistry.

The composition may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In one embodiment, the composition comprises the pharmaceutically acceptable salt of cytarabine, 1-(β-D-arabino-furanosyl)-cytosine hydrochloride. In another embodiment, the composition comprises the pharmaceutically acceptable salt of CPX, ciclopirox olamine.

In yet another embodiment, the composition includes CPX and/or cytarabine prodrugs. In general, such prodrugs will be functional derivatives of CPX and cytarabine which are readily convertible in vivo into the compound from which it is notionally derived. Prodrugs of CPX and cytarabine may be conventional esters formed with the available hydroxy. For example, the available OH in CPX or cytarabine may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the application are those in which one or more of the hydroxy groups in the compounds are masked as groups which can be converted to hydroxy groups in vivo. Conventional procedures for the selection and preparation of suitable prodrugs are described, for example, in “Design of Prodrugs” ed. H. Bundgaard, Elsevier, 1985.

CPX and an effective amount of cytarabine are suitably formulated into pharmaceutical compositions for administration to human subjects in a biologically compatible form suitable for administration in vivo. Accordingly, the present application further includes a pharmaceutical composition comprising CPX and an effective amount of cytarabine and a pharmaceutically acceptable carrier and/or diluent.

The application in one aspect, also describes a pharmaceutical composition comprising an effective amount of CPX and an effective amount of cytarabine and a pharmaceutically acceptable carrier for treatment of leukemia in a subject in need of such treatment.

The compositions described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences. On this basis, the compositions include, albeit not exclusively, solutions of the substances in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween®), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesyl-phosphotidyl-ethanolamine (DOPE), and liposomes. Such compositions should contain a therapeutically effective amount of the compound, together with a suitable amount of carrier so as to provide the form for direct administration to the patient.

The compositions described herein can be administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer. Wherein the route of administration is oral, the dosage form may be for example, incorporated with excipient and used in the form of enteric coated tablets, caplets, gelcaps, capsules, ingestible tablets, buccal tablets, troches, elixirs, suspensions, syrups, wafers, and the like. The dosage form may be solid or liquid.

Accordingly in one embodiment, the application describes a pharmaceutical composition wherein the dosage form is a solid dosage form. A solid dosage form refers to individually coated tablets, capsules, granules or other non-liquid dosage forms suitable for oral administration. It is to be understood that the solid dosage form includes, but is not limited to, non-controlled release, controlled release and time-controlled release dosage form units, employed suitably in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet.

Compositions suitable for buccal or sublingual administration include tablets, lozenges, and pastilles, wherein the active ingredient is formulated with a carrier such as sugar, acacia, tragacanth, or gelatin and glycerin.

In another embodiment the application describes a pharmaceutical composition wherein the dosage form is a liquid dosage form. A liquid dosage form refers to non-solid dosage forms suitable for, but not limited to, intravenous, subcutaneous, intramuscular, or intraperitoneal administration. Solutions of CPX and cytarabine described herein can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999.

Sustained or direct release compositions can be formulated, e.g. liposomes or those wherein the active compound is protected with differentially degradable coatings, such as by microencapsulation, multiple coatings, etc. It is also possible to freeze-dry the compounds of the application and use the lyophilates obtained, for example, for the preparation of products for injection.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

In one embodiment the dosage form comprises about 20 mg to 2000 about mg of CPX and an effective amount of cytarabine. In another embodiment, the dosage form comprises about 50 mg to about 500 mg of CPX and an effective amount of cytarabine. The dosage form may alternatively comprise about 40 to about 500 mg, about 250 to about 500 mg, about 1 to about 200 mg of CPX/kg body weight, about 5 to about 50 mg of CPX/kg body weight, about 10 to about 40 mg of CPX/kg body weight or about 25 mg of CPX/kg body weight of a subject in need of such treatment.

A further aspect is a composition, wherein the oral dosage form is selected from enteric coated tablets, caplets, gelcaps, and capsules, comprising from about 20 to less than 1000 mg, suitably from about 50 to about 500 mg, of CPX and an effective amount of cytarabine.

An effective amount of cytarabine includes for example, amounts approved by the FDA. For example, doses of cytarabine can range from about 10 mg per day subcutaneously (low dose) to about 6000 mg twice daily by bolus intravenous infusion (high dose). In one embodiment the effective amount is about 100 mg/m2 by continuous intravenous infusion daily for up to for example 7 days.

Another aspect provides a commercial package comprising a composition described herein, and associated therewith instructions for the use thereof for treatment of a leukemic disorder such as acute myeloid leukemia or acute lymphoid leukemia in a subject in need of such treatment. In another embodiment, the commercial package is for the treatment of chronic myelogenous leukemia, lymphoma or multiple myeloma. Another embodiment provides a commercial package comprising a composition described herein, and associated therewith instructions for the inducing cell death and/or inhibiting survivin activity or level in a leukemic disorder cell such as a leukemia cell.

The following non-limiting examples are illustrative of the present application:

### EXAMPLES

### Materials and Methods

### Cell Culture

Leukemia (HL-60, RSV411, k562, Jurkat, U937), LYMPHOMA (MDAY-D2), solid tumour cell lines (PPC-1, HeLa, OVCAR-3, DU-145, HT-29) and GMO5757 human lung fibroblasts were cultured in RPMI 1640 medium. HepG2 hepatoma cells and MRC5 human lung fibroblasts were grown in Dulbecco modified Eagle medium. OCI-M2, OCI-AML2 and NB4 leukemia cell lines and OPM2, KMS11, LP1, UTMC2, KSM18 and OCIMy5 myeloma cell lines were maintained in Iscove Modified Dulbecco Medium. LF1 human lung fibroblasts were maintained in HAM medium. All media were supplemented with 10% fetal calf serum, 100 μg/mL of penicillin, and 100 units/mL of streptomycin (all from Hyclone, Logan, Utah). The cells were incubated at 37° C. in a humidified air atmosphere supplemented with 5% CO₂.

### Reagents

All the following reagents were purchased from Sigma (St Louis, Mo.): Ciclopirox olamine, Deferoxamine mesylate, 1-(β-D-Arabino-furanosyl)-cytosine hydrochloride (AraC) and Daunorubicin.

### Cloning and Transfections

The full-length survivin promoter (−1059 upstream of the initiating ATG) (NIEHS-SNPs, Environmental Genome Project, NIEHS ES15478, Department of Genome Sciences, Seattle, Wash.) was isolated from HeLa genomic DNA using the forward primer 5′-GGCGAGCTCACTTTTTCTGTCACCTCCGTGGTCCG-3′ (SEQ ID NO:1) and the reverse primer 5′-GGGTTCGAAACGGCGGCGGCGGTGGAGA-3′ (SEQ ID NO:2).

The promoter was sub-cloned into the GL4.20 firefly luciferase reporter vector (Promega Corporation, Madison, Wis.). Clones were sequence-verified for orientation and integrity using a CEQ 8000 Genetic Analysis System (Beckman, Mississauga, ON, Canada).

HeLa cells were transfected with surviving promoter construct alone or vector alone using Lipofectamine (Invitrogen, CA) according to the manufacturer's instructions and stable clones selected with Puromycin (4 μg/ml) (Sigma).

### Identification of Inhibitors of Survivin Transactivation

(A) HeLa cells stably overexpressing the survivin promoter driving luciferase (15000 cells/well) were plated in 96-well plates. After adhering to the plates, cells were treated with aliquots of chemicals from the LOPAC (Sigma), Prestwick (Prestwick Chemical, Illkirch, France), Spectrum (Microsource, Gaylordsville, Conn.) and Biomol (BIOMOL International L.P., Plymouth Meeting, Pa.) libraries of off patent drugs, chemicals and natural products. The final concentration of compounds was 5 μM (0.05% dimethyl sulfoxide [DMSO]). Cells were incubated with the molecules for 24 hours. After incubation, survivin promoter activity was assessed by the luciferase assay described below. Results were normalized and corrected for systematic errors using the B score (J. Biomol. Screen. 8, 624-633). Compounds with a B score value lower than 3 times the standard deviation were empirically considered hits in the assay.

Plate handling was performed by a CRS Dimension4 robotics platform equipped with a Linear Plate Transport system (LPT) (Thermo Electron, MA). Plate transfer from the LPT to online peripherals was carried out by a CRS Flip Mover and Vertical Array Loader (Thermo Electron). Liquid handling steps were performed by a Biomek FX Laboratory Automation Workstation (Beckman Coulter) and ELx405 Magna cell washers (Biotek, Vermont). Robotics integration was controlled by a Polara integration software (Thermo Electron).

### Luciferase Assay

Luciferase activity was measured according to the manufacturer's instructions and as previously described (Promega, Madison, Wis.) (Mao Blood). In brief, the cell culture medium was removed using an Embla plate washer (Molecular Devices, Sunnyvale, Calif.) and 1× Glo Lysis buffer (Promega) was added by the robotic liquid handler. After 10-minutes incubation, an equal volume of Bright-Glo Luciferase substrate (Promega) was added, and the luminescence signal was detected with a 96-well Luminoskan luminescence plate reader (Thermo Fisher Scientific, Waltham, Mass.) with 5-seconds integration time.

### Viability Assays

The CellTiter96 aqueous nonradioactive (MTS) assay was used to measure cell viability according to the manufacturer's instructions (Promega). Propidium iodide (PI) staining was used according to manufacturer's instructions (Biovision, Mountain view, Calif.).

### Quantitative Real-Time Polymerase Chain Reaction

cDNAs encoding survivin and glyceraldehyde-3-phosphate dehydrogenase (GAPDH) were amplified using the following primer pairs: survivin, forward, 5′-TTTTCATCGTCGTCCCTAGC-3′ (SEQ ID NO:3); reverse, 5′-CGACTCAGATGTGGCAGAAA-3′ (SEQ ID NO:4); and GAPDH, forward, 5′-GAAGGTGAAGGTCGGAGTC-3′ (SEQ ID NO:5); reverse, 5′-GAAGATGGTGATGGGATTTC-3′ (SEQ ID NO:6). Equal amounts of cDNA for each sample were added to a prepared master mix (SYBR Green PCR Master mix; Applied Biosystems, Foster City, Calif.). Quantitative real-time polymerase chain reaction (RTPCR) reactions were performed on an ABI Prism 7700 sequence detection system (Applied Biosystems, Foster City, Calif.). The relative abundance of a transcript was represented by the threshold cycle of amplification (C_{T}), which is inversely correlated to the amount of target RNA/first-strand cDNA being amplified. To normalize for equal amounts of the latter, the transcript levels of the putative housekeeping gene GAPDH were assayed. The comparative C_{T} method was calculated by the manufacturer's instructions. The expression level of survivin relative to the baseline level was calculated as 2^{-ΔΔC}^{_{T}}^{(survivin)}, where ΔCT is (average survivin C_{T}−average GAPDH C_{T}) and is ΔΔCT (average ΔCT-untreated sample−average ΔCT-treated sample).

### Immunoblotting

Total cell lysates were prepared from cells. Briefly, cells were washed with phosphate-buffered saline pH 7.4, and suspended in lysis buffer (10 mmol/L Tris, pH 7.4, 150 mmol/L, NaCl, 0.1% Triton X-100, 0.5% sodium deoxycholate, and 5 mmol/L EDTA) containing protease inhibitors (Complete tablets; Roche, Ind.). Nuclear extracts were isolated after a cytoplasm protein extraction by incubating the cells with the cytoplasm buffer on ice for 15 minutes (10 mM HEPES, 10 mM KCL, 0.1 mM EDTA, 0.1 mM EGTA, DTT 1 mM, NP40 0.65%, protease inhibitors, pH=7.4) and centrifugation at 4° C. for 1 minute at 10000 g. The pellet was suspended in the lysis buffer for 30 minutes (10 mmol/L Tris, pH 7.4, 150 mmol/L, NaCl, 0.1% Triton X-100, 0.5% sodium deoxycholate, SDS 1.7%, glycerol 5% and 5 mmol/L EDTA) for 30 minutes and then centrifuged at 4° C. at maximum speed for 20 minutes. Protein concentrations were measured by the Bradford assay (Anal Biochem. 1996; 236:221-228). Equal amounts of protein were subjected to sodium dodecyl sulphate (SDS)-polyacrylamide gels followed by transfer to polyvinylidene difluoride membranes.

Membranes were probed with polyclonal rabbit anti-human survivin (1 μg/mL) (NOVUS), monoclonal mouse anti-human p53 (0.5 μg/mL), polyclonal rabbit anti-human GR (0.5 μg/mL) both from (Santa Cruz Biotechnologies, CA), or with mouse anti-human GADPH (Trevigen, Gaithersburg, Md.). Secondary antibodies (GE Healthcare, Chalfont St Giles, United Kingdom) were horseradish peroxidase-conjugated goat anti mouse IgG (1:10 000 [v/v]) and anti rabbit (1:5000 [v/v]). Detection was performed by the enhanced chemical luminescence method (Pierce, Rockford, Ill.).

### Cell Cycle

Cell-cycle analysis was performed as previously described (Blood 112(3):760). Briefly, cells were harvested, washed with cold PBS, re-suspended in 70% cold ethanol, and incubated overnight at −20° C. Cells were then treated with 100 ng/mL of DNase-free RNase (Invitrogen, Carlsbad, Calif.) at 37° C. for 30 minutes, washed with cold PBS, and re-suspended in PBS with 50 μg/mL of PI (Sigma). DNA content was analyzed by flow cytometry (FACSCalibur; BD Biosciences, San Jose, Calif.).

### Assessment of CPX Anticancer Activity in Mouse Models of Leukemia

MDAY-D2 (MDAY) murine leukemia cells (5×10⁵) were injected intra-peritoneal into NOD/SCID mice (Ontario Cancer Institute, Toronto, ON), OCI-AML2 and K562 cells (2×10⁶) were injected subcutaneously into both flanks of sub-lethally irradiated (3.5 Gy) NOD/SCID mice. Mice were then treated daily by oral gavages with CPX (25 mg/kg) in PBS or vehicle control. Tumour volume (tumour length×width²×0.5236) (Mol Cancer Ther. 2004; 3:1239-1248) was measured weekly using calipers. Eight (MDAY-D2), 11 (OCI-AML2) or 30 (K562) days after injection of cells, mice were sacrificed, and the volume and weight of the tumours was measured. All animal studies were carried out according to the regulations of the Canadian Council on Animal Care and with the approval of the local ethics review board.

### Statistical Analysis

Results were expressed as mean plus or minus SD. Treatment effects were compared using Student t test, and differences between means were considered to be significant when p was less than 0.05. All in vitro experiments were repeated at least 3 times. Calcusyn software (Biosoft, UK) was used to analyze drug combination data.

### A High Throughput Screen Identifies Ciclopirox

CPX (ciclopirox olamine) was identified in a high throughput screen for inhibitors of the survivin promoter. Survivin is over-expressed in leukemia cells and primary patient samples (2). Therefore, inhibitors of surviving were sought. To identify such small molecule inhibitors, a high throughput chemical genomics screen was developed. HeLa cells stably over-expressing the human survivin promoter driving firefly luciferase were exposed to the compound libraries (n=4,800) at 5 μM for 24 hours and luciferase expression was tested. Forty five compounds (0.94%) reduced luciferase expression and these compounds were retested in a secondary screening for reproducibility (luciferase assay) and viability (MTS assay). The same incubation time using the same system resulted in 43 compounds corroborating the luciferase reduction (91.1%) and 16 preferentially decreasing luciferase over viability (35.5%).

From this chemical screen, the antimicrobial CPX was identified. CPX is currently used for the topical treatment of cutaneous fungal infections. As an anti-fungal agent, the mechanism of action of CPX is not well understood, but appears related to binding intracellular iron and inhibiting iron containing enzymes. To explore its efficacy and mechanism of action as an anti-cancer agent, leukemia cell lines were treated with increasing concentrations of CPX. 72 hours after incubation, cell viability was measured by the MTS assay. CPX decreased cell viability in all leukemia cell lines tested and decreased cell viability in 5/9 leukemia cell lines with an LD₅₀<5 μM (FIG. 2; a concentration that is pharmacologically achievable based on prior animal studies investigating CPX as an anti-fungal). Cell death was confirmed by the presence of a subG1 peak by flow cytometry after staining cells with propidium iodide. In contrast, CPX was less toxic to MRC 5, LF1, and GMO 5757 non-malignant fibroblasts with an LD₅₀>20 μM.

To further evaluate the therapeutic potential of CPX, CPX was investigated in combination with cytarabine and Daunorubicin, chemotherapeutic agents commonly used for the treatment of acute myeloid leukemia. Here, OCI-AML2 leukemia cells were treated with CPX alone and in combination with cytarabine and Daunorubicin at 4, 2, 1, 0.5 and 0.25 times their LD₅₀. Seventy-two hours later, viability was assessed by MTS assay. Data was analyzed by the Calcusyn median effect model where the combination index (CI) indicates synergism (CI<0.9), additively (CI=0.9-1.1) or antagonism (CI>1.1). The combination between CPX and cytarabine demonstrated a strong synergism with CI values at the ED50, ED75 and ED90 of 0.18, 0.19 and 0.24 respectively (FIG. 3). In contrast, the combination with CPX and daunorubicin was closer to additive with CI values at the ED50, ED75 and ED90 of 0.85, 0.88 and 1.2, respectively.

Given the effects in leukemia cells lines, the effects of oral CPX was evaluated in 3 mouse models of leukemia. Sublethally irradiated NOD-SCID mice were injected subcutaneously with OCI-AML2 or K562 human leukemia cells or intraperitoneally with MDAY-D2 murine leukemia cells. After tumor implantation, mice were treated with CPX (25 mg/kg) in water or water alone by oral gavage. Oral CPX decreased tumor weight and volume in all 3 mouse models by up to 65% compared to control without evidence of weight loss or gross organ toxicity (FIG. 4).

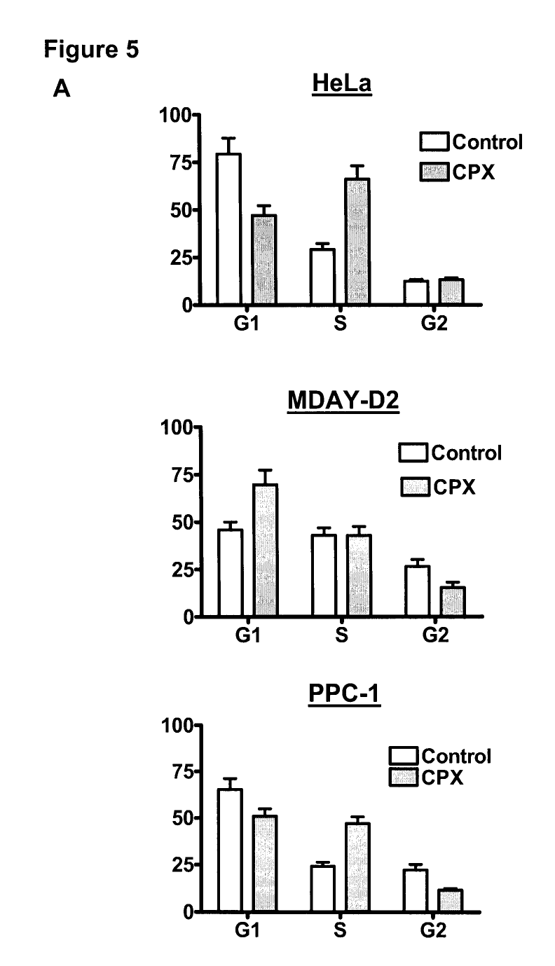

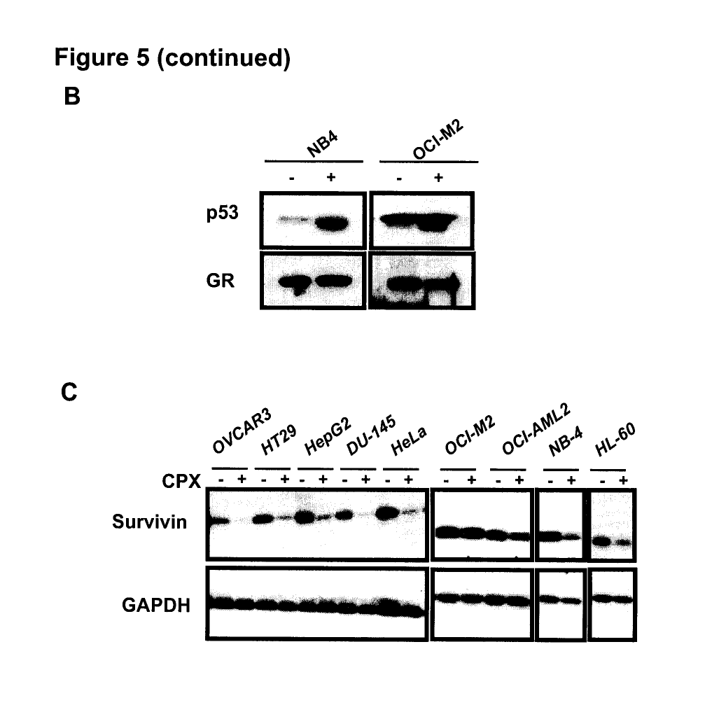

Mechanistically, CPX arrested cells in the G1/S phase of the cell cycle and down-regulated the expression of survivin, Cyclin D1, and the transcription factors YY1 and FTII-D prior to the onset of cell death (FIG. 5).

### Discussion

Leukemic disorders such as acute myeloid leukemia (AML) and acute lymphoid leukemia (ALL) are malignant diseases resulting in the proliferation of abnormal cells of myeloid and lymphoid origin, respectively. Leukemic disorders are characterized by poor responses to standard therapies. It would be advantageous for these patients and those with relapsed refractory disease if novel therapies were available. As many of these patients are frail, therapies that achieve an anti-leukemia effect without significant toxicity are highly desirable.

CPX alone and in combination with cytarabine was found to induce cell death in malignant leukemia cell lines.

### Example 2

### Mouse Xenograft Model

Sublethally irradiated NOD-SCID mice are inoculated subcutaneously in the flanks with OCI-AML2, MDAY-D2 and U937 leukemia cells. The CPX and cytarabine co-treatment is initiated when tumors reach volumes of 200 mm³ at which time mice are randomized to receive 50 mg/kg of CPX and an effective amount of cytarabine (treated group) or buffer control (untreated group) for 5 to 7 days. Caliper measurements are performed twice weekly to estimate tumor volume and differences compared between treated and untreated groups.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Adida C, Haioun C, Gaulard P, et al. Prognostic significance of survivin expression in diffuse large B-cell lymphomas. Blood 2000; 96:1921-5.
2. Tamm I, Richter S, Oltersdorf D, et al. High expression levels of x-linked inhibitor of apoptosis protein and survivin correlate with poor overall survival in childhood de novo acute myeloid leukemia. Clin Cancer Res 2004; 10:3737-44.
3. Mao X, Zhu X, Hurren R, Ezzat S, and Schimmer A D. Dexamethasone increases ubiquitin transcription through an SP-1 dependent mechanism in multiple myeloma cells. Leuk Res 2008.
4. Mawji I A, Simpson C D, Gronda M, et al. A chemical screen identifies anisomycin as an anoikis sensitizer that functions by decreasing FLIP protein synthesis. Cancer Res 2007; 67:8307-15.
5. Mao X, Liang S B, Hurren R, et al. Cyproheptadine displays preclinical activity in myeloma and leukemia. Blood 2008; 112:760-9.

1. A method of treating a leukemic disorder comprising administering an effective amount of ciclopirox and an effective amount of cytarabine to a subject in need of such treatment.

2. The method according to claim 1 wherein said leukemic disorder selected from leukemia, lymphoma, multiple myeloma and a refractory malignancy.

3. The method according to claim 2 wherein said leukemia is acute myeloid leukemia, chronic myelogenous leukemia or acute lymphoid leukemia.

4. The method according to claim 3 wherein said acute myeloid leukemia is a high-risk acute myeloid leukemia or wherein said acute lymphoid leukemia is a high-risk acute lymphoid leukemia.

5-7. (canceled)

8. The method as claimed in claim 1, wherein said effective amount of ciclopirox is within a range of about 1 to about 200 mg/kg body weight or within a range of about 5 to about 50 mg/kg body weight.

9. The method as claimed in any one of claims 1 to 7, wherein said effective amount of ciclopirox is within the range of about 5 to about 50 mg/kg body weight.

10. The method of claim 1, wherein said effective amount of ciclopirox and said effective amount of cytarabine are administered as a pharmaceutical composition in a dosage form.

11. The method of claim 10, wherein said dosage form is suitable for oral administration or injection.

12. (canceled)

13. The method of claim 11, wherein said dosage form contains from about 20 mg to about 1000 mg of said ciclopirox and an effective amount of cytarabine or from about 40 mg to about 500 mg of said ciclopirox and an effective amount of cytarabine.

14-15. (canceled)

16. A pharmaceutical composition for treatment of a leukemic disorder comprising an effective amount of ciclopirox, an effective amount of cytarabine and a pharmaceutically acceptable carrier in a dosage form, wherein said dosage form contains from about 20 mg to about 1000 mg of said ciclopirox and an effective amount of cytarabine, from about 40 mg to about 500 mg of said ciclopirox and an effective amount of cytarabine, an amount suitable to provide about 1 to about 200 mg of ciclopirox/kg body weight or an amount suitable to provide about 5 to about 50 mg of ciclopirox/kg body weight, formulated into a solid oral dosage form, a liquid dosage form or an injectable dosage.

17. (canceled)

18. The pharmaceutical composition of claim 16, which is suitable for oral administration or for injection.

19-21. (canceled)

22. The composition as claimed in claim 16, wherein the amount of ciclopirox is effective for treatment of acute myeloid leukemia, acute lymphoid leukemia or chronic myelogenous leukemia.

23. The composition as claimed in claim 16, wherein the oral dosage form is selected from enteric coated tablets, caplets, gelcaps and capsules.

24. The composition as claimed in claim 16, comprising from about 20 to about 1000 mg of ciclopirox or from about 50 to about 500 mg of ciclopirox.

25-27. (canceled)

28. A commercial package about comprising a composition according to claim 16, and associated therewith instructions for the use thereof for treatment of acute myeloid leukemia or acute lymphoid leukemia in a subject in need of such treatment.

29-33. (canceled)

34. The method of claim 1 for inducing cell death in a leukemic disorder cell comprising administering an effective amount of ciclopirox and cytarabine.

35. A method of inhibiting survivin activity or levels comprising detecting increased activity or level of survivin in a leukemic disorder cell or population of leukemic disorder cells compared to a control; and contacting the cell or cells with ciclopirox and cytarabine, wherein the ciclopirox and cytarabine are contacted in a combined amount effective to decrease the activity or level of survivin.

36. The pharmaceutical composition of claim 16 wherein the leukemic disorder is selected from leukemia, lymphoma, multiple myeloma and a refractory malignancy.

37. The method, of claim 1 wherein the ciclopirox comprises ciclopirox olamine and/or the cytarabine comprises 1-(β-D-arabino-furanosyl)-cytosine hydrochloride.

38. The method, of claim 1 wherein the cytarabine comprises 1-(β-D-arabino-furanosyl)-cytosine hydrochloride.

39. The composition of claim 16, wherein the ciclopirox comprises ciclopirox olamine and/or wherein the cytarabine comprises 1-(β-D-arabino-furanosyl)-cytosine hydrochloride.
